(19) United States
(12) Patent Application Publication
(10) Pub. No.: US 2013/0064889 A1
(43) Pub. Date: Mar. 14, 2013

(54) Tablet-in-tablet Palperidone Formulations and Methods for Production and Use Thereof

(71) Applicants: Siva Rama Krishna Nutalapati, Princeton, NJ (US); Rakeshkumar Khushalbhai Lad, Marlton, NJ (US)

(72) Inventors: Siva Rama Krishna Nutalapati, Princeton, NJ (US); Rakeshkumar Khushalbhai Lad, Marlton, NJ (US)

(73) Assignee: Aptapharma, Inc., Pennsauken, NJ (US)

(21) Appl. No.: 13/608,045

(22) Filed: Sep. 10, 2012

Related U.S. Application Data
(60) Provisional application No. 61/533,900, filed on Sep. 13, 2011.

(51) Int. Cl. A61K 9/24; A61K 31/519; A61P 25/22; A61P 9/06; B05D 5/00; A61P 7/02; A61P 25/18; A61P 35/00; A61P 25/06; A61K 9/28; A61P 31/04

(52) U.S. Cl. 424/465; 514/259.41; 427/2.21

(57) ABSTRACT

Tablet-in tablet paliperidone formulations and processes for preparation and use thereof are provided.

This patent application claims the benefit of priority from U.S. Provisional Application Ser. No. 61/533,900, filed Sep. 13, 2011, the teachings of which are hereby incorporated by reference in their entirety.

### FIELD OF THE INVENTION

The present invention relates to tablet-in tablet pharmaceutical formulations comprising paliperidone or a salt thereof, and processes for preparation and use thereof.

### BACKGROUND OF THE INVENTION

Paliperidone (CAS Registry No. 144598-75-4) has the chemical name 4H-Pyrido[1,2-a]pyrimidin-4-one, 3-[2-[4-(6-fluoro-1,2-benzisoxazol-3-yl)-1-piperidinyl]ethyl]-6,7,8,9-tet-rahydro-9-hydroxy-2-methyl and is a major human metabolite of the known antipsychotic drug risperidone and is itself an antipsychotic. Paliperidone has been disclosed in U.S. Pat. No. 5,158,952 (EP0368388). Subsequently an amorphous and several crystalline forms of paliperidone were disclosed in WO2008/021342.

Paliperidone has been approved for short-term treatment of acute schizophrenia and for long-term or maintenance treatment of schizophrenia. It is marketed as an extended release tablet under the brand name INVEGA® (Ortho-McNeil-Janssen Pharmaceuticals, Inc.). The tablets are based on an osmotic delivery technology, wherein osmotic pressure is used to deliver paliperidone at a controlled rate. The package insert for the U.S. INVEGA® tablet describes the tablet as a “delivery system, which resembles a capsule-shaped tablet in appearance, [which] consists of an osmotically active trilayer core surrounded by a subcoat and semipermeable membrane. The trilayer core is composed of two drug layers containing the drug and excipients, and a push layer containing osmotically active components. There are two precision laser-drilled orifices on the drug-layer dome of the tablet. Each tablet strength has a different colored water-dispersible overcoat and print markings. In an aqueous environment, such as the gastrointestinal tract, the water-dispersible color overcoat erodes quickly. Water then enters the tablet through the semipermeable membrane that controls the rate at which water enters the tablet core, which, in turn, determines the rate of drug delivery. The hydrophilic polymers of the core hydrate and swell, creating a gel containing paliperidone that is then pushed out through the tablet orifices. The biologically inert components of the tablet remain intact during gastrointestinal transit and are eliminated in the stool as a tablet shell, along with insoluble core components.”

WO 2004/010981 (EP 1539115 and published U.S. Application No. 2004/0092534) describes an osmotic dosage form for controlled delivery of paliperidone. The dosage form delivers paliperidone for more than about 22 hours and exhibits a substantially ascending rate of release with 90% delivery occurring at about 20 hours.

WO 2006/017537 (EP 1802286) discloses a sustained dosage form having an ascending zero order release pattern. The dosage form structure comprises a delay layer comprising a polymeric matrix and a microencapsulated drug; and a second layer comprising a polymeric matrix and non-microencapsulated drug matrix. The delay layer is substantially free of non-microencapsulated drug and the second layer is located adjacent to the delay layer. Example 3 describes a paliperidone tablet containing an inner core surrounded by an inner layer and an outer layer end caps. Microencapsulated paliperidone is contained in the inner core and inner layer but not in the outer layer. The trilayer arrangement is achieved by compressing the inner layer and outer layer material simultaneously onto the compressed inner core; e.g. sometimes referred to in the art as compression coating or press coating.

WO 2006/085856 discloses dosing with benzisoxazole derivatives such as paliperidone in order to achieve certain blood plasma parameters. In general, for a single dose “D” the following two criteria are desired: (a) a mean dose proportional maximum plasma concentration of 0.5 ng/mL/mg<Cₘₐₓ/D<8 ng/mL/mg and (b) a mean dose proportional area under a plasma concentration-time curve of 30 ng.hr/mL/mg<AUC_{inf}/D<300 ng.hr/mL/mg, or, exhibiting the time of the peak plasma concentration Tₘₐₓ>9 hours. A variety of osmotic dosage forms are described for achieving the desired blood plasma criterion, though the application indicates it is not limited to osmotic dosage forms.

WO 2007/044234 and WO 2007/081736 disclose osmotic dosage forms having both controlled release and fast release aspects. Both patent applications disclose examples of paliperidone-containing osmotic dosage forms.

WO2009/025859 discloses a paliperidone sustained release composition having a first and second component. The first component comprises a delay layer comprising a polymer and the second component comprises non-coated paliperidone. The first and second components are adjacent to each other and in particular can be arranged as an “inlay” tablet. The inlay tablet comprises an inlay core containing non-coated paliperidone and at least one water swelling polymer. Partially surrounding the inlay core is an outer layer that contains a water insoluble pharmaceutical excipient. Preferably the tablet has a relative bioavailability of 1.5 to 3.0 times the bioavailability of the commercial INVEGA® tablet.

As the osmotic system employed in the commercial INVEGA® tablet is somewhat complicated to manufacture, it would be desirable to find a simpler system to provide controlled release of paliperidone, preferably a system that is able to be bioequivalent to INVEGA®.

WO 2011/018246 discloses a tablet-in-tablet design for controlled release of paliperidone comprising an inner hydrogel tablet core comprising paliperidone or a pharmaceutically acceptable salt thereof and an outer hydrogel layer surrounding the inner tablet core, wherein the outer layer does not contain paliperidone or a pharmaceutically acceptable salt thereof.

There remains a need for alternative controlled release paliperidone formulations.

### SUMMARY OF THE INVENTION

The present invention relates to the discovery of a tablet-in-tablet design for controlled release of paliperidone.

An aspect of the present invention relates to a controlled-release pharmaceutical tablet, comprising an inner tablet comprising paliperidone or a pharmaceutically acceptable salt thereof and an outer tablet surrounding the inner tablet and comprising paliperidone or a pharmaceutically acceptable salt thereof, said inner tablet and/or outer tablet being optionally coated with a subcoating and/or a functional coating.

Another aspect of the present invention relates to a method for producing the above tablet. The method comprises compression coating an outer tablet comprising paliperidone or a pharmaceutically acceptable salt thereof around an inner tablet comprising paliperidone or a pharmaceutically acceptable salt thereof, to form a tablet-in-tablet formulation for controlled release of paliperidone. In one embodiment, the tablet-in tablet formulation is optionally coated with a functional coating or film. In this embodiment, the tablet-in tablet formulation may be coated with a subcoating prior to the functional coating or film.

Yet another aspect of the present invention relates to a method for the treatment of paliperidone-treatable diseases, e.g., schizophrenia, by administering a tablet of the present invention to a patient in need thereof.

### DETAILED DESCRIPTION OF THE INVENTION

The present invention relates to tablet-in-tablet formulations for controlled release of paliperidone as well as methods for their production and use in treating paliperidone-treatable diseases, e.g., schizophrenia. In the tablet-in-tablet formulations of the present invention, both the inner and outer tablets contain paliperidone.

A “tablet-in-tablet” design means that the dosage form comprises an inner tablet that is covered and surrounded by an outer coat, also referred to herein as outer tablet, which is compressed onto the inner tablet. Both inner and outer tablets are made by a compression process that is characteristic for making tablets, hence the “tablet-in-tablet” expression. Tablet presses allowing such a technique are known as alternate tablet presses, “tablet-in tablet” presses, or dry coat tablet presses, and are known in the art.

For purposes of the present invention, by “paliperidone” as used herein, it is meant to refer to paliperidone per se (i.e., paliperidone base) as well as its pharmaceutically acceptable salts. Generally the total amount of paliperidone in the formulation is from 1 to 15 mg, and frequently 1.5 mg to 12 mg, (expressed in terms of the weight of the paliperidone base in the case of a salt thereof). Paliperidone is not microencapsulated in the formulations of the present invention.

In the tablet-in-tablet formulations of the present invention, the inner tablet comprising paliperidone is compression coated with an outer tablet comprising paliperidone.

The amount of paliperidone in the inner tablet and the outer tablet can be in the ratios of about 4:1 to about 1:4, specifically about 3:1 to about 1:3. As will be understood by the skilled artisan upon reading this disclosure, however, alternative ratios can be used to modify the drug release profiles from these tablets.

In addition to paliperidone, the inner tablet further comprises a release retarding excipient. Examples of release retarding excipients include, but are not limited to, hydrophilic polymers such as hydroxypropylmethylcellulose, hydroxymethylcellulose, hydroxypropylcellulose and hydroxyethylcellulose, and which swell in contact with aqueous liquids, and control release of the drug by diffusion through the swollen polymer network. Examples of other release retarding excipients include, but are not limited to, waxes such as carnauba wax, bees wax, stearic acid and gums such as acacia, acrylic polymers, shellac, zein, polyvinylpyrrolidine including crosslinked polyvinylpyrrolidinone, vinyl acetate copolymers, polyethylene oxides, polyvinyl alcohols, and combinations comprising at least one of the foregoing materials.

The inner tablet may further comprise additional excipients such as binders, diluents and/or lubricants. In general, the inner tablet contains at least one compressible matrix-forming binder/filler excipient. In one embodiment, the compressible matrix-forming binder/filler excipient is a hydrophilic compound that may be water soluble or water insoluble. Examples of such binder/fillers include, but are not limited, microcrystalline cellulose(s), silicified microcrystalline cellulose(s), polyvinylpyrrolidone, starch, hydroxypropyl cellulose and combinations thereof. A binder/filler generally constitutes from 10 to 70%, more typically 25 to 65% of the weight of the inner tablet.

The inner tablet may also comprise a soluble filler or fillers used to facilitate the solubility of the drug substance in the matrix, if necessary, and support the diffusion of drug substance via the matrix network, if needed.

In addition, the inner tablet may further comprise a buffering agent which can modify the solubility of paliperidone in the intestinal tract. Examples of such buffering agents include organic or inorganic acids and/or bases. In one embodiment, tartaric acid or fumaric acid is used as the acid and magnesium oxide is used as the base.

The inner tablet may further comprise a glidant for better flow of powders during tabletting. A nonlimiting example of an appropriate glidant(s) is colloidal silicon dioxide.

In addition, the inner tablet may comprise a lubricant to avoid adherence of powders to the punches. Nonlimiting examples of suitable lubricants include magnesium stearate and sodium stearyl fumarate.

In addition to paliperidone, the outer tablet further comprises a release retarding material. Examples of release retarding excipients include, but are not limited to hydrophilic polymers such as hydroxypropylmethylcellulose, hydroxymethylcellulose, hydroxypropylcellulose and hydroxyethylcellulose, and which swell in contact with aqueous liquids, and control release of the drug by diffusion through the swollen polymer network. Examples of other release retarding excipients include, but are not limited to, waxes such as carnauba wax, bees wax stearic acid and gums such as acacia, acrylic polymers, shellac, zein, polyvinylpyrrolidine including crosslinked polyvinylpyrrolidinone, vinyl acetate copolymers, polyethylene oxides, polyvinyl alcohols, and combinations comprising at least one of the foregoing materials.

The outer tablet may further comprise additional excipients as described above for the inner tablet, such as, but not limited to binders, fillers, lubricants, and combinations thereof. In one embodiment, the excipient(s) are selected to increases the elastic properties and allow for adhesion of the outer tablet to the inner tablet via compression coating. Usually at least one binder/filler such as described above for the inner tablet is present in the outer tablet. The outer tablet may further comprise a glidant and/or lubricant as described above for the inner tablet.

The formulations of the present invention are capable of releasing low doses (e.g., 1-15 mg) of paliperidone over a prolonged period. The release pattern or dissolution curve is determined in part by the amount of paliperidone in each tablet or layer and/or by the amount and type of release retarding agent. In addition, the mass ratio of the inner tablet:outer tablet has an influence on the release rate. In general, the mass ratio of inner tablet to outer tablet is at least 1:2, respectively, typically in the range from 1:1.5 to 1:8, and in some embodiments preferably from 1:4 to 1:6.

From a practical perspective the total mass of the inner tablet is generally within the range of 40 to 100 mg, more typically 50 to 70 mg. The mass of the outer tablet is generally within the range of 150 to 480 mg, more typically 240 to 360 mg, including about 280, 300, 320, or 340 mg. The inner tablet and the outer tablet are normally the same shape, preferably round including flat round or a convex round tablet shape. The inner tablet usually has a diameter of 7 millimeters or less, usually 6 millimeters or less. The outer tablet preferably has a diameter of about 11 millimeters or less, typically 9 to 10.5 millimeters and in some embodiments about 10 millimeters.

In some embodiments the inner tablet and the outer tablet may be of different shapes, one being, for example, round and the other being, for example, oval or caplet shape.

In one embodiment, the tablet-in-tablet dosage form of the present invention is coated with a functional coating or film. By “functional coating or film” it is meant a coating that modifies the release properties of the formulation. Examples of such coatings or films include, but are not limited to, controlled release, delayed release, modified release, pH dependent, pH independent coatings, and any combinations thereof.

In one embodiment, the inner tablet of the tablet-in-tablet formulation of the present invention is coated with a functional coating.

In one embodiment, both the inner tablet and the outer tablet of the tablet-in-tablet formulation of the present invention are coated with functional coating. Examples of functional coating materials include, but are not limited to, film forming polymers such as an alkylcellulose including methylcellulose or ethylcellulose, a hydroxyalkylcellulose such as hydroxymethylcellulose, hydroxyethylcellulose, hydroxypropylcellulose and hydroxybutylcellulose, a hydroxyalkyl alkylcellulose such as hydroxyethyl methylcellulose and hydroxypropyl methylcellulose, a carboxyalkylcellulose such as carboxymethylcellulose, an alkali metal salt of carboxyalkylcelluloses such as sodium carboxymethylcellulose, a carboxyalkyl alkylcellulose such as carboxymethyl ethylcellulose, a carboxyalkylcellulose ester, a starch, a pectin such as sodium carboxymethylamylopectine, a chitin derivate such as chitosan, a polysaccharide such as alginic acid, alkali metal and ammonium salts thereof, a carrageenan, a galactomannan, traganth, agar-agar, gum arabicum, guar gum and xanthan gum, acrylic acid, polyacrylic acid and the salts thereof, a polyvinylalcohol, a polyvinylpyrrolidone, a copolymer of polyvinylpyrrolidone with vinyl acetate, a polyalkylene oxide such as polyethylene oxide and polypropylene oxide and a copolymer of ethylene oxide and propylene oxide, or a combination comprising at least one of the foregoing.

Examples of enteric polymers include, but are not limited to, polymers such as methacrylic acid-ethyl acrylate copolymer (1:1), ethacrylic acid-methyl methacrylate copolymer (1:1), methacrylic acid-methyl methacrylate copolymer (1:2), polyvinyl acetate phthalate (PVAP), hydroxypropyl methylcellulose acetate succinate (HPMCAS) and cellulose acetate phthalate (CAP). Additionally, dyestuffs or pigments can be added to the enteric polymer coating for product identification or to characterize the quantity of active compound, i.e., dosage.

The functional coating may optionally comprise a plasticizer, an additional film-former, a pore former, or a combination comprising at least one of the foregoing.

The pore-forming agents used are preferably water soluble materials. Examples of pore-forming materials include, but are not limited to, polymers like hydroxyalkyl celluloses such as hydroxymethyl cellulose, hydroxyethyl cellulose, hydroxypropyl cellulose and hydroxybutylcellulose, hydroxyalkyl alkylcelluloses such as hydroxyethyl methylcellulose and hydroxypropyl methylcellulose, polyvinylalcohols, polyvinylpyrrolidones, copolymers of polyvinylpyrrolidone with vinyl acetate, sugars, salts and combinations thereof.

In some embodiments, prior to applying the functional coating or film, the tablet-in-tablet formulation is coated with a subcoating and then coated with the functional coating or film to avoid interactions of the paliperidone with the functional coating or film. Examples of subcoating materials include, but are not limited to, film forming polymers like hydroxyalkyl celluloses such as hydroxymethyl cellulose, hydroxyethyl cellulose, hydroxypropyl cellulose and hydroxybutylcellulose, hydroxyalkyl alkylcelluloses such as hydroxyethyl methylcellulose and hydroxypropyl methylcellulose, polyvinylalcohols, polyvinylpyrrolidones, copolymers of polyvinylpyrrolidone with vinyl acetate, and combinations thereof.

The tablet-in-tablet formulation of the present invention can provide release of paliperidone for more than 18, and preferably more than 20 hours, as measured in an in vitro dissolution test. The dissolution testing is carried out in 500 ml of phosphate buffer at pH 6.8 in a Type I apparatus at 100 rpm paddle speed. Typically, the preferred tablet of the present invention exhibits the following dissolution profile expressed in terms of the percentage amount of paliperidone released at various durations:

Less than 25% in 6 hours;

Less than 55% in 12 hours, preferably 25-50%; and

Less than 100% in 20 hours, preferably 85-98%.

Some embodiments will meet only one or two of the release requirements for the three time points listed. Additionally, some tablet embodiments of the present invention are able to provide a substantially S-shaped dissolution curve. Such a curve is characterized in that the release rate is relatively slow in the first time period of release then it is relatively high for a significant portion of the release profile, before the release rate slows thereby forming an “S” shaped curve. It is desirable that the S-shaped dissolution curve be relatively long and not a step-function. For example, the time from release of 20% of the paliperidone to the time of release of 80% of the paliperidone is desirably at least 5 hours, more preferably at least 6 hours, and in some embodiments at least 8 hours such as 8 to 14 hours.

Some formulation embodiments of the present invention have a dissolution curve similar to that of the INVEGA® and/or are considered bioequivalent in vivo to INVEGA®.

In one embodiment, the tablet-in-tablet formulation further comprises one or more additional orally active agents. Any orally active agent can be included in the tablet-in-tablet formulation. Examples include, but are not limited to, alpha-2 adrenergic agents, analgesics, angiotensin-converting enzyme (ACE) inhibitors, antianxiety agents, antiarrhythmics, antibacterials, antibiotics, anticoagulants, anticonvulsants, antidepressants, antidiabetics, antiemetics, antiepileptics, antifungals, antihelminthics, antihistamines, antihyperlipidemics, antihypertensives, antiinfectives, antimalarials, antimicrobials, antimigraine agents, antimuscarinic agents, antineoplastic agents, antiprotozoals, antipsychotics, antispasmodics, antiretroviral agents, antivirals, attention-deficit hyperactivity disorder (ADHD) agents, β-blockers, calcium channel blockers, chemotherapeutic agents, cholinesterase inhibitors, Cox-2 inhibitors, decongestants, diuretics, histamine-2 receptor antagonists, hypnotics, hypoglycemic agents, hypotensive agents, immunosuppressants, lipotropics, neuroleptics, opioid analgesics, peripheral vasodilators/vasoconstrictors, proton pump inhibitors, sedatives, serotonin receptor agonists, sympathomimetics as well as pharmaceutically acceptable salts, solvates, hydrates, stereoisomers (racemates, individual enantiomers or diastereomers, or any combination thereof), or polymorphs thereof, or pharmaceutically acceptable combinations comprising at least one of the foregoing active agents, and the like. In this embodiment, an additional active agent or agents may be included in the inner tablet, the outer tablet or both the inner and outer tablet. The same additional active agent or agents may be included in the inner and outer tablets or different active agents may be included in the inner and outer tablets.

The formulations of the present invention can be made by conventional tablet-in-tablet or compression coating techniques. The inner tablet is first made via any convenient tabletting technique such as direct compression or wet granulation, though direct compression is preferred for simplicity when the excipient selection so permits. A tablet blend, which contains the ingredients of the inner tablet including paliperidone and a release retarding material is compressed in a tablet punch to form a tablet; e.g., a round tablet of diameter 6 mm. This tablet forms the inner tablet of the final “tablet-in-tablet” formulation. Then a second, larger tablet punch, e.g., round diameter 10 mm, is partially charged with a portion of outer tablet mixture comprising paliperidone and a release retarding material. The previously produced inner tablet is then placed and centered in the partially charged punch and additional outer tablet mixture comprising paliperidone and the release retarding material is added and the whole material compressed to form a compression coating outer tablet around the inner tablet; e.g., a tablet-in-tablet. Tablet presses allowing such a technique are known as alternate tablet presses, “tablet-in tablet” presses, or dry coat tablet presses, and are known in the art.

In one embodiment, the inner tablet of the tablet-in-tablet formulation is coated with a functional coating or film.

In one embodiment, the both inner tablet and outer tablet of the tablet-in-tablet formulation are coated with a functional coating or film.

In one embodiment, the tablet-in-tablet formulation is coated with a functional coating or film.

Examples of such coatings or films include, but are not limited to, controlled release, delayed release, modified release, pH dependent, pH independent coatings, and any combinations thereof. The functional coating may optionally comprise a plasticizer, an additional film-former, a pore former, or a combination comprising at least one of the foregoing.

In some embodiments, prior to applying the functional coating or film, the core tablet is coated with a subcoating or non-functional coating and then coated with the functional coating or film to avoid interactions of the API with the functional coating or film.

The tablet in-tablet formulations of the present invention can be used in a method for treating schizophrenia and other diseases treatable by paliperidone and/or for making medicaments for treating the same. Such methods comprise administering an effective amount of the tablet-in-tablet formulation to a patient in need thereof. The tablets may be administered in dosage amounts and regimens corresponding to those known and recommended in the art.

The following nonlimiting examples are provided to further illustrate the present invention.

### EXAMPLES

### Example 1

### Tablet in a Tablet Formulation of Paliperidone

| | mg/tab |
|---|---|
| Inner Tablet | |
| Paliperidone | 4.2 |
| Carnauba Wax, NF | 20.6 |
| Stearic Acid, NF | 12 |
| Hydroxy Propylcellulose, NF (Klucel HXF) | 12 |
| Silicon Dioxide, NF | 0.7 |
| Magnesium Stearate, NF | 0.5 |
| Total weight | 50 |
| Outer Tablet | |
| Paliperidone | 1.8 |
| Lactose Monohydrate, NF | 80 |
| Microcrystalline cellulose, NF (Avicel PH200) | 101 |
| Polyvinyl pirrolidone, NF (Plasdone K29/32) | 15 |
| Hydroxy Propylmethylcellulose, NF | 60 |
| (Hypromellose K100 LVCR) | |
| Magnesium Stearate, NF | 2.2 |
| Total weight | 260 |

Inner Tablet: Stearic acid is dissolved in SD3A alcohol by warming up to 50° C. All other ingredients except Silicon Dioxide and Magnesium Stearate are dry mixed in a high shear granulator. The mixture is wet granulated with Stearic acid solution to form a wet granulation mixture. The wet granulation mixture is screened, dried, and milled. The milled granules, Silicon Dioxide and Magnesium Stearate are then blended in a blender. The final blend is then compressed into 5 mm tablets of 50 mg each.

Outer Tablet: Plasdone K29/32 is dissolved in water. All other ingredients except Magnesium Stearate are dry mixed in a high shear granulator. The mixture is wet granulated with Plasdone solution to form a wet granulation mixture. The wet granulation mixture is screened, dried, and milled. The milled granules and Magnesium Stearate are then blended in a blender.

Compression of Tablet in a Tablet: Approximately 130 mg of the outer tablet blend was added to the die cavity. The inner tablet is dispensed and centered into the die cavity. Remaining 130 mg of the outer tablet blend was added to the die cavity and the outer tablet was compressed using Kikusui Aquarius LD tablet press.

### Example 2

### Tablet in a Tablet Formulation of Paliperidone

| | mg/tab |
|---|---|
| Inner Tablet | |
| Paliperidone | 4 |
| Polyvinyl pirrolidone, NF (Plasdone K29/32) | 6 |
| Hydroxy Propylmethylcellulose, NF | 10 |
| (Hypromellose K100 MCR) | |
| Lactose Monohydrate, NF | 10 |
| Microcrystalline cellulose, NF (Avicel PH200) | 14 |
| Glyceryl behenate | 5 |
| Silicon Dioxide, NF | 0.5 |
| Magnesium Stearate, NF | 0.5 |
| Total weight | 50 |
| Outer Tablet | |
| Paliperidone | 2 |
| Lactose Monohydrate, NF | 80 |
| Microcrystalline cellulose, NF (Avicel PH200) | 101 |
| Polyvinyl pirrolidone, NF (Plasdone K29/32) | 15 |
| Hydroxy Propylmethylcellulose, NF | 60 |
| (Hypromellose K100 LVCR) | |
| Magnesium Stearate, NF | 2 |
| Total weight | 260 |

Inner Tablet: Plasdone K29/32 is dissolved in water. All other ingredients except Silicon Dioxide and Magnesium Stearate are dry mixed in a high shear granulator. The mixture is wet granulated with Plasdone solution to form a wet granulation mixture. The wet granulation mixture is screened, dried, and milled. The milled granules, Silicon Dioxide and Magnesium Stearate are then blended in a blender. The final blend is then compressed into 5 mm tablets of 50 mg each.

Outer Tablet: Plasdone K29/32 is dissolved in water. All other ingredients except Magnesium Stearate are dry mixed in a high shear granulator. The mixture is wet granulated with Plasdone solution to form a wet granulation mixture. The wet granulation mixture is screened, dried, and milled. The milled granules and Magnesium Stearate are then blended in a blender.

Compression of Tablet in a Tablet: Approximately 130 mg of the outer tablet blend was added to the die cavity. The inner tablet is dispensed and centered into the die cavity. Remaining 130 mg of the outer tablet blend was added to the die cavity and the outer tablet was compressed using Kikusui Aquarius LD tablet press.

### Example 3

### Tablet in a Tablet Formulation Coated with Surelease

The tablet in a tablet formulations of Examples 1-2 can be coated with an extended release coating material (Surelease) containing an optional pore former.

| | % per |
|---|---|
| Ingredient | batch |
| Surelease ® | 80 |
| Opadry Clear | 20 |
| Water* | qs |

*Removed in process

Add Opadry Clear to water and mix until a clear solution is obtained. To this add Surelease and mix for 45 minutes. Continue mixing while spraying. The tablet in a tablet cores are added into a perforated coating pan or a fluid bed with a Wurster insert. The coating suspension is sprayed onto the cores. A coating level of about 5-25% coat weight is applied.

### Example 4

### Tablet in a Tablet Formulation Coated with Eudragit

The tablet in a tablet formulations of Examples 1-2 can be coated with an extended release coating material (Eudragit RS/RL or Eudragit L100) containing an optional pore former.

| | % per |
|---|---|
| Ingredient | batch |
| Eudragit RS | 60 |
| Eudragit RL | 12 |
| Triethyl citrate | 8 |
| Talc | 20 |
| SD3A alcohol* | qs |

*Removed in process

Add Eudragit RS and Eudragit RL to SD3A alcohol and mix until a clear solution is obtained. To this add Triethyl citrate and mix for 30 minutes. Add Talc and mix for 45 minutes. Continue mixing while spraying. The tablet in a tablet cores are added into a perforated coating pan or a fluid bed with a Wurster insert. The coating suspension is sprayed onto the cores. A coating level of about 5-25% coat weight is applied.

### Example 5

### Inner Tablet of the Tablet in a Tablet Formulation Coated with Eudragit

The inner tablet of Examples 1-2 can be coated with an extended release coating material (for example Eudragit RS/RL) or with an enteric coating material (for example Eudragit L100) or with any other release modifying material before compressing into tablet in a tablet formulation.

| | % per |
|---|---|
| Ingredient | batch |
| Eudragit L100 | 72 |
| Triethyl citrate | 8 |
| Talc | 20 |
| SD3A alcohol* | qs |

*Removed in process

Add Eudragit L100 to SD3A alcohol and mix until a clear solution is obtained. To this add Triethyl citrate and mix for 30 minutes. Add Talc and mix for 45 minutes. Continue mixing while spraying. The inner tablet cores are added into a perforated coating pan or a fluid bed with a Wurster insert. The coating suspension is sprayed onto the cores. A coating level of about 5-25% coat weight is applied. The tablet in a tablet cores can be compressed as explained in Examples 1 and 2.

What is claimed is:

1. A controlled-release pharmaceutical tablet, comprising an inner tablet comprising paliperidone or a pharmaceutically acceptable salt thereof and a release retarding excipient within an outer tablet comprising paliperidone or a pharmaceutically acceptable salt thereof and a release retarding excipient.

2. The controlled release pharmaceutical tablet of claim 1 wherein the paliperidone or pharmaceutically acceptable salt thereof in the inner tablet and/or outer tablet is not microencapsulated.

3. The controlled release pharmaceutical tablet of claim 1 further comprising a functional coating or film surrounding the outer tablet.

4. The controlled release pharmaceutical tablet of claim 3 further comprising a subcoating between the outer tablet and functional coating or film.

5. The controlled release pharmaceutical tablet of claim 1 further comprising a functional coating or film surrounding the inner tablet.

6. The controlled release pharmaceutical tablet of claim 5 further comprising a subcoating between the inner tablet and functional coating or film.

7. The controlled release pharmaceutical tablet of claim 1 wherein the amount of paliperidone in the inner tablet and the outer tablet is at a ratio of about 4:1 to about 1:4.

8. The controlled release pharmaceutical tablet of claim 1 wherein the amount of paliperidone in the inner tablet and the outer tablet is at a ratio of about 3:1 to about 1:3.

9. The controlled release pharmaceutical tablet of claim 1 comprising an additional active agent or agents in the inner tablet, the outer tablet or the inner and our tablet.

10. A method for producing the controlled-release pharmaceutical tablet of claim 1, said method comprising compression coating an outer tablet comprising paliperidone or a pharmaceutically acceptable salt thereof and a release-retarding excipient around an inner tablet comprising paliperidone or a pharmaceutically acceptable salt thereof and a release retarding excipient.

11. The method of claim 10 wherein the paliperidone or pharmaceutically acceptable salt thereof in the inner tablet and/or outer tablet is not microencapsulated.

12. The method of claim 10 further comprising coating the outer tablet with a functional coating or film.

13. The method of claim 12 further comprising applying a subcoating between the outer tablet and functional coating or film.

14. The method of claim 10 further comprising coating the inner tablet with a functional coating or film.

15. The method of claim 14 further comprising applying a subcoating between the inner tablet and functional coating or film.

16. A method for treating a paliperidone-treatable disease in a patient in need thereof, said method comprising administering to the patient the controlled-release pharmaceutical tablet of claim 1.
